(19) United States
(12) Patent Application Publication
(10) Pub. No.: US 2015/0004262 A1
(43) Pub. Date: Jan. 1, 2015

(54) ANESTHETIC AND THERAPEUTIC COMPOSITION FOR USE IN AQUACULTURE

(71) Applicant: KUWAIT INSTITUTE FOR SCIENTIFIC RESEARCH, Safat (KW)

(72) Inventors: AMANI AL-YAQOUT, Salmiya (KW); AZAD ISMAIL SAHEB, Salmiya (KW); MUSAAD AL-ROUMI, Salmiya (KW); MOHAMMED AL-KANDARI, Salmiya (KW)

(21) Appl. No.: 14/489,287

(22) Filed: Sep. 17, 2014

Related U.S. Application Data
(62) Division of application No. 13/568,235, filed on Aug. 7, 2012.

(51) Int. Cl. A61K 36/54; A61K 36/53

(52) U.S. Cl. CPC A61K 36/54; A61K 36/53
USPC 424/739

(57) ABSTRACT

An anesthetic and therapeutic composition for use in aquaculture includes an essential oil preferably thyme oil that is mixed with ethanol in an amount of one part essential oil preferably thyme oil to five parts ethanol. The essential oil ethanol mixture is then dispersed in water preferably seawater to provide 10 to 60 ppm of essential oil. The oxygen in the composition is between about 5.00 and 6.5 mg/L and in a preferred embodiment is enhanced by an agar stabilizer. The invention also contemplates treating fish such as sheam and sobaity and shrimp and seahorse to reduce stress and maintain a temperature of about 20°-27° C.

### FIELD OF THE INVENTION

This invention relates to an anesthetic and therapeutic composition for use in aquaculture and to a method for treating fish and shrimp for reducing stress and as a therapeutic for bacterial and parasitic infections.

### BACKGROUND FOR THE INVENTION

The principle use of anesthetics in fish husbandry and management is to minimize stress during routine procedures such as handling the fish during capturing, transportation, sorting, measurements or tagging. Such manipulations have strong effects on both physiology and suppress immunological capacity. It has been recognized that an ideal anesthetic should provide rapid immobility, ease in handling, non-toxicity to fish, no mammalian safety problems, low tissue residues and low costs.

A second problem related to the fish industry namely fish husbandry and management is to counter the effects of different types of bacteria that are responsible for a wide spectrum of diseases reported among aquatic organizations. Antibiotics have been the main therapeutics used in most aquaculture systems for the control of bacterial diseases and infections. Regular and prolonged use of antibiotics results in the development of antibiotic resistance among the potential pathogenic bacteria. This continues to be a major health concern worldwide.

To be more specific the ciliated parasitic protozoan, Uronema sp. are considered as one of the most serious parasitic threats faced by marine aquaculturists over the world. They cause extensive skin sloughing, hemorrhage, damage to the skin, gill epithelium and musculature. They are known for systemic infection destroying vital organs and fish tissue leading to significant mortalities. The candidate victims for these parasites are cultured silver pomfret (Pampas argenteus) and seahorses. The sub-adult zobaidy suffered 55%-70% mortalities due to scuticociliatosis by Uronema sp. Several chemicals were applied to control Uronema sp. such as daily baths for 60 minutes or more in 100 ppm of formaldehyde which is classified as a known human carcinogen.

The handling of aquatic animals both in and out of their natural environment almost always involves physical activity. Stress-related cortisol released in fish can suppress immunological capacity. Anesthetics are agents used to induce, first, a calming effect, then successive loss of equilibrium, mobility, consciousness, and finally, reflex action in an organism exposed to higher concentrations of the drug, or exposed for a longer time. An ideal anesthetic chemical should fulfill some criteria like rapid immobility, ease in handling, non-toxicity to fish, no mammalian safety problems, low tissue residues, and low cost. Furthermore, the behavioral responses of the organism to it should be simple, and in addition to a calm induction, safe and rapid recovery from an anesthetic is required.

Organic farming of agricultural and horticultural crops is being used as a popular venture in the direction of sustained and eco-friendly food production. Organic farming will look for alternatives to those chemicals that are currently being used in aquaculture, and the anesthetics are one such important input. As a result, different chemical anesthetics were investigated to compare their effectiveness with a natural product, known as clove oil, on different species of fish.

Aromatic plants and their extracted essential oils have been used traditionally for a wide variety of purposes, their antibacterial, antifungal, antioxidant and anticarcinogenic properties, make them suitable for many applications in food preservation, food additives, folk medicine and pharmaceuticals. In recent years, there has been an interest in the use of natural substances prompting more detailed studies on plant derivatives. Inhibitory activity of these substances against the growth of several microorganisms such as fungi, parasites and viruses has been reported. The hydrophobicity of essential oils (Eos) enables them to partition in the lipids of the cell membrane and mitochondria, rendering them permeable and leading to leakage of cell contents. Essential oils (EOs) are natural antimicrobials with potential to extend the shelf-life of seafood when used alone or in combination with other preservation techniques.

Thyme (Thymus volgaris) oil is being used traditionally to treat hysteria, indigestion, colic, to promote menstruation, and for the control of fever. The EO of thyme is made up of 20-55% thymol, a powerful antiseptic for both internal and external use. Thyme oil is employed as a local anesthetic in human medicine, and before modern antibiotics were developed, it was used to medicate bandages. Oil of thyme is the important commercial product obtained by distillation of fresh leaves and flowering tops of T. vulgaris. It is extensively used in processed food. The Greeks used thyme as an antiseptic. Thyme can be used in food systems to prevent the growth of food-borne bacteria and extend the shelf life of processed foods. Thyme oil was found to be very effective with a lowest minimum inhibitory concentration (MIC) of 1.25% (v/v) against Bacillus sp., Listeria monocytogenes, E. coli and Klebsiella sp. Amongst the fungi, Rhizomucor sp. was found to be highly sensitive to the oil. Thyme essential oil exhibited antibacterial activities against 25 microorganisms; also, it showed inhibition of Shigella sp. and bactericidal effect. Toxigenic fungi were found to be sensitive to thyme leading to inhibition of fungal growth, and mycotoxin production was dependent on the concentration of EOs used. Thyme oil can also be used as antimicrobial coating on the shelf life of the precooked shrimp, Penaeus sp.

During the investigation in the project, “Eco-Friendly Essential Oils of Plant Origin as Antipathogenic Agents in Fish Health Management” (FM055C), thyme (Thymus vulgaris) oil was found to exhibit qualities of an anesthesia against sheam, Acanthopagrus latus, and to confirm this effect another study has been completed FM068K and the results confirmed this anesthetic effect on sheam, sobaity fish and shrimp. There are no studies known to Applicant's about the anesthetic effect of thyme oil on any of the marine or fresh water fish.

Oregano and thyme EO, particularly when enhanced by agar stabilizer, may be effective in reducing the number or preventing the growth of E. coli O157:H7 in foods. Oregano exhibited considerable inhibitory effects against all the organisms under test while their major components demonstrated various degrees of growth inhibition. It has been reported that preservation using oregano oil can extend the shelf-life of seafood through inhibiting the specic spoilage organism P. phosphoreum.

Cinnamaldehyde in cinnamon oil has been reported to inhibit five of the bacteria involved in meat spoilage. Maqbool et al. has reported that concentration up to 0.3% can be used for extending the storage life of bananas for up to 28 days without affecting the physico-chemical properties. Using 500 or 1000 ppm of cinnamon oil as a dietary additive for Broilers chicks has showed that cinnamon oil has hypocholesterolaemic and antioxidant characteristics, and it also improved meat quality.

Sage has been proven to have antibacterial effect.

A search of U.S. patents for use of essential oils disclosed a U.S. patent of McCue et al. No. 5,403,587 which discloses a disinfectant and sanitizing composition based on essential oil. For example, the patent discloses an aqueous antimicrobial composition containing one or more essential oils in a water carrier. The reference teaches the use of an essential oil obtained from thyme to sanitize, disinfect and clean hard surfaces.

An additional U.S. Pat. No. 5,629,281 of Butler teaches the use of herbal oils including thyme to relieve pain of minor headaches by a topical application to the underside of the wrist.

Further, a U.S. patent of Carnevali U.S. Pat. No. 6,391,323 discloses a composition having analgesic, antiseptic and skin healing promoting activity for the treating of burns, sunburn, scalds, irritation, sores and abrasions. The compound includes an active ingredient namely chlorophyll, cod liver and camphor, preferably together with a natural antiseptic agent such as thyme oil.

In addition, a U.S. Patent Application Publication No. 2003/0059480 of Boratyn discloses compositions and methods relating to fish milt and intra-lamellar gels from algae as skin and hair products. Here the publication suggests adding an essential oil such as thyme oil to the intra-lamellar gel.

Finally, the search disclosed a U.S. Pat. No. 7,368,135 of Anderson for a herbal healing oil as a liquid topical analgesic to reduce pain from sore muscles, bruised ligaments, lower back pain, etc.

### BRIEF SUMMARY OF THE INVENTION

In essence, an anesthetic and therapeutic composition for use in aquaculture comprises or consists of an essential oil selected from the group consisting of thyme oil, cinnamon oil, oregano oil, sage oil, garlic oil, eucalyptus oil, clove oil and mixtures thereof. The composition also includes a mass of ethanol and wherein the essential oil is mixed with ethanol in an amount of one part essential oil to five parts ethanol and wherein the essential oil and alcohol is added to a tank of water, particularly seawater, to provide 10 to 120 ppm of essential oils.

In the composition of the preferred embodiment of the invention, the essential oil is thyme oil and the desired oxygen in the anesthetic and therapeutic composition is between about 5.00 and 6.5 mg/l and in which the thyme oil is enhanced by an agar stabilizer.

The invention also comprises or consists of a method for treating fish, sheam and shrimp with an anesthetic for reducing stress through a routine procedure and as a therapeutic for bacteria and parasitic infections. The method comprises or consists of providing a first tank and a mass of seawater disposed in the first tank. The first tank of seawater is maintained at a temperature of between about 20° C. and 27° C. An essential oil selected from the group comprising or consisting of thyme oil, cinnamon oil, oregano oil and mixtures thereof and ethanol and wherein the mixture provides a ratio of one part essential oil to five parts ethanol and wherein the amount of essential oil is between 10 ppm and 120 ppm essential oil and preferably between 10 ppm and 60 ppm. The method further includes a second tank and mass of seawater disposed therein and maintained at a temperature between about 20° C. and 27° C.; exposing a fish to the essential oil in the first tank for a period of about ten minutes and thereafter removing the fish from the first tank and placing the fish in the second tank and allowing it or them to remain therein for 96 hours of observations and detection of any mortality. Finally, the fish are tested for changes in hemotology (HGB, HCT, NEUT, LYMPH, MONO, EO, Cortisol and Glucose).

Further, in a preferred embodiment of the method for treating fish and shrimp in which the essential oil is thyme oil.

### DESCRIPTION OF THE DRAWINGS

FIG. 1 is the mean of the effects of different concentrations of essential oils and anesthetics on sheam fingerlings;

FIG. 2 is the mean of recovery from different concentrations of essential oils and anesthetics on sheam fingerlings;

FIG. 3 illustrates the anesthetic effect thyme oil on sub-adult of sheam (Acanthopagrus latus) fish;

FIG. 4 illustrates the need of effects of different concentrations of thyme oil as an anesthetic and total recovery time of sheam (Acanthopagrus latus);

FIG. 5 shows the sleep time of essential oils on sobaity (Sparidentex hasta) fingerlings;

FIG. 6 illustrates the total recovery time of essential oils on sobaity (Sparidentex hasta) fingerlings;

FIG. 7 shows the effect of different concentrations of thyme oil (ppm) on total anesthesia and recovery time of sobaity (Sparidentex hasta) fingerlings;

FIG. 8 illustrates the effect of different concentrations of thyme oil (ppm) on total anesthesia and recovery time of sobaity (Sparidentex hasta) fingerlings and sub-adult;

FIG. 9 illustrates the tissue residues in fish treated with 10 ppm and 20 ppm of essential oils;

FIG. 10 shows the serum lysozyme levels (Units/ml) in treated fish against different anesthetics;

FIG. 11 is a comparison of serum protein levels (g/100 ml) in treated fish against different anesthetics;

FIG. 12 illustrates the effect of a Sobaity surface swab total bacterial and vibrio (log₁₀) counts (TBC and TVC) and their percent inactivation after treatment with thyme oil;

FIG. 13 illustrates a Shaem surface swab total bacterial and vibrio (log 10) counts (TBC and TVC) and their percent inactivation after treatment with thyme oil;

FIG. 14 illustrates the cinnamon oil effect on V. anguillarum;

FIG. 15 illustrates the cinnamon oil effect on V. parahaemolyticus;

FIG. 16 illustrates the cinnamon oil effect on V. harveyi;

FIG. 17 illustrates the bacterial inhibition of the essential oils;

FIG. 18 illustrates the bacterial inhibition of the essential oils;

FIG. 19 illustrates the bacterial inhibition of the essential oils;

FIG. 20 illustrates the bacterial inhibition of the essential oils;

FIG. 21 illustrates the bacterial inhibition of the essential oils;

FIG. 22 illustrates the bacterial inhibition of the essential oils;

FIG. 23 illustrates the bactericidal activity of the essential oils (as determined by micro drop agar plating method) against different pathogenic bacteria;

FIG. 24 illustrates the bactericidal activity of the essential oils (as determined by micro drop agar plating method) against different pathogenic bacteria;

FIG. 25 illustrates the bactericidal activity of the essential oils (as determined by micro drop agar plating method) against different pathogenic bacteria;

FIG. 26 illustrates the bactericidal activity of the essential oils (as determined by micro drop agar plating method) against different pathogenic bacteria;

FIG. 27 illustrates the bactericidal activity of the essential oils (as determined by micro drop agar plating method) against different pathogenic bacteria;

FIG. 28 illustrates the bactericidal activity of the essential oils (as determined by micro drop agar plating method) against different pathogenic bacteria;

FIG. 29 illustrates the effect of different essential oils versus time on Uronema sp.;

FIGS. 30A-30C illustrate bacterial eye infection in Shaeim (Acanthopagrus latus) showing healing effects of natural oils before treatment, during treatment, and after treatment, respectively.

FIGS. 31A-31J illustrate stages of cell depredation following exposure to most of the EOs tested.

Similar reference characters denote corresponding features consistently throughout the attached drawings.

### DESCRIPTION OF THE PREFERRED EMBODIMENTS OF THE INVENTION

### I. Anesthesia

Thyme oil (Thymus vulgaris), cinnamon oil (Cinnamomum zeylanicum), oregano oil (Origanum vulgare), clove oil (Syzygium aromaticum), were mixed in a ration 1:5 with ethanol. Effects of different concentrations of all treatments on the tested fish were monitored until 10 min and any behavioral changes in the fish were noted. The dose was considered effective if the fish lose their equilibrium into total sleep within 10 min. Time of total sleep and complete recovery from the anesthesia were recorded. Once anaesthetized, the fish were shifted to normal rearing conditions for recording observations for a 96 hour period to record any mortality or any change in feeding behavioral. Fish were used only once and then returned to the holding tanks.

### Experiment (1)

### Anesthetic Effect of Thyme, Cinnamon and Oregano Oils Tested on Fingerlings of Sheam (Acanthopagrus latus) Fish at 23° C.

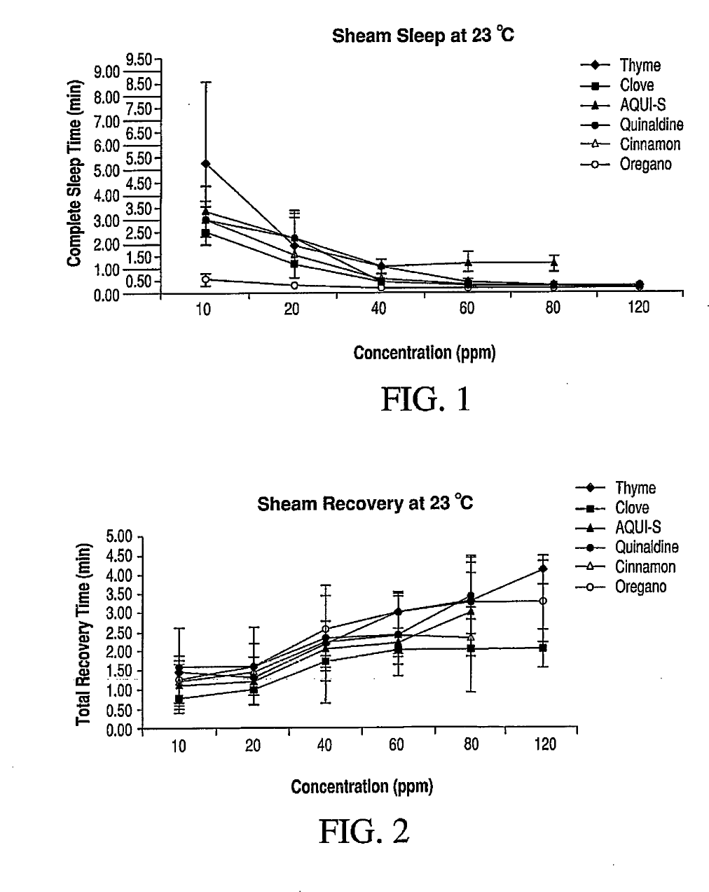

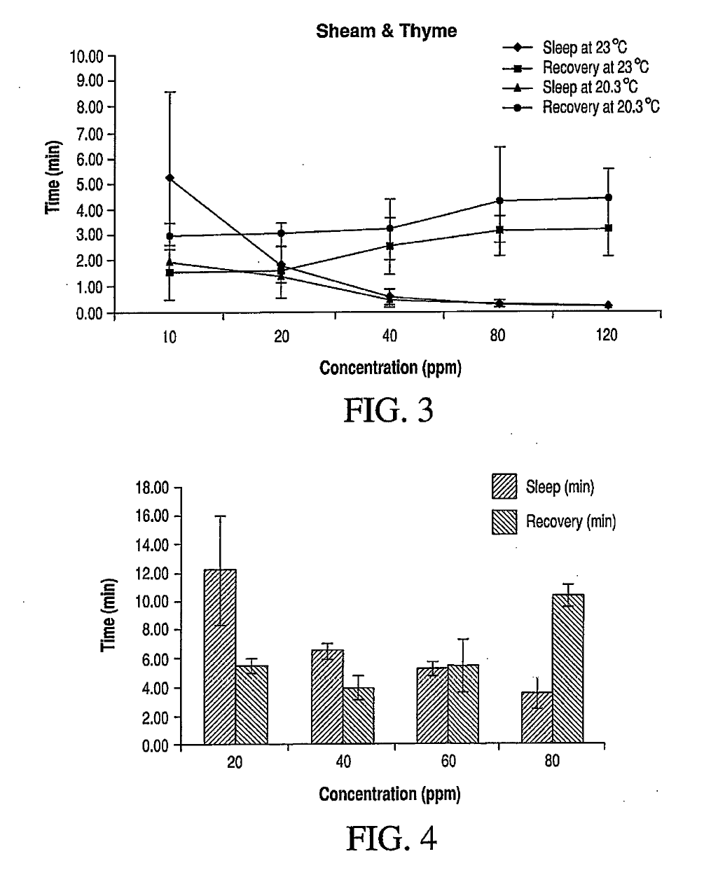

### NOTE: FIGS. 1, 2 and 3

The tested concentrations were (10, 20, 40, 60, 80 and 120 ppm) of the selected E0s and chemicals. The fish weight was 16.70±8.30 g. The data showed that all the used concentrations of all EOs were effective and there was a correlation between the concentration and time elicited to reach deep sleep, as the concentration increase, the time decreases. There was no mortality observed in the concentrations between 10 to 120 ppm even after 96-hours post-recovery from thyme, oregano and cinnamon essential oil treatments. The dissolved oxygen (DO) in the anesthetic and recovery chambers was between 5.00 and 6.00 mg/l. Thyme seemed to fulfill the criteria for the ideal anesthetic like rapid immobility, ease in handling, non-toxicity to fish, no mammalian safety problems, and low tissue residues.

TABLE 1
Stages of Anaesthetization and Recovery in Fish Fingerlings

| Stage | Description | Behavior |
|---|---|---|
| 1 | Normal | Reactive to external stimuli; opercular rate |
| | | and muscle tone normal. |
| 2 | Start of anesthetic | Partial loss of muscle tone; swimming erratic; |
| | induction | increased opercular rate; reactive |
| | | only to strong tactile and vibrational stimuli. |
| 3 | Total sleep | Total loss of reactivity; opercular movements |
| | | slow and irregular; loss of all Reflexes |
| 4 | Start of recovery | Partial recovery of equilibrium with partial |
| | | recovery of swimming motion. |
| 5 | Complete recovery | Reappearance of avoidance in swimming |
| | | motion and reaction in response to external |
| | | stimuli, but still, behavioral response is stolid. |

### Experiment (2)

### Anesthetic Effect of Thyme Oil Tested on Fingerlings of Sheam (Acanthopagrus latus) Fish at 23° C. And 20.5° C.

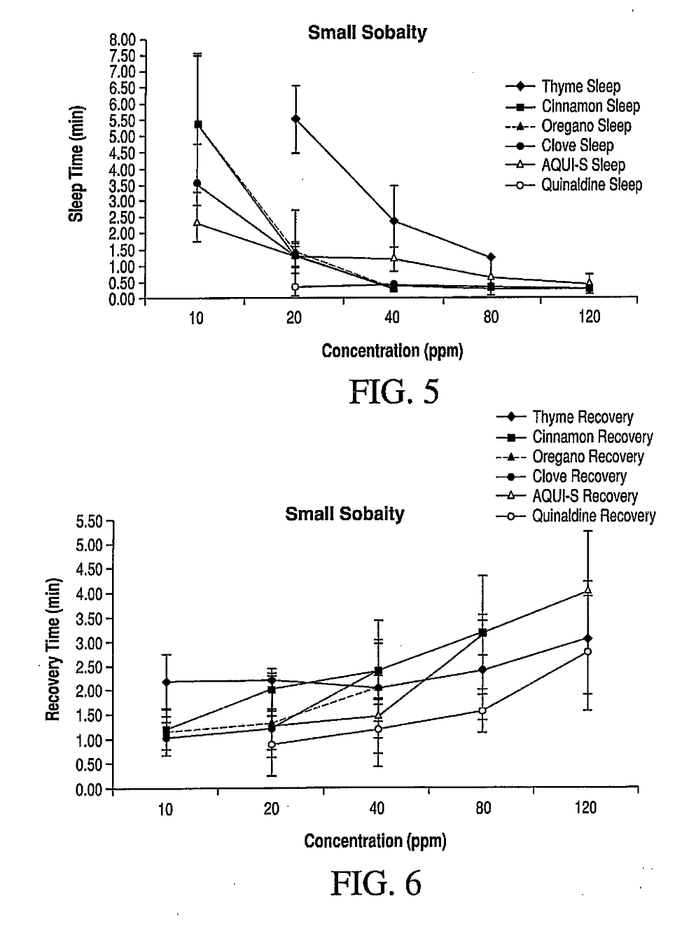

With reference to FIG. 5, 20 fish of sheam were tested for the concentrations 10, 20, 40, 80 and 120 ppm at two temperatures 23° C. and 20.5° C. The tested fish weight was 13.10±1.18 g at 23° C. and 10.08±0.20 g at 20.5° C.

### Experiment (3)

### Anesthetic Effect of Thyme Oil Tested on Sub-Adult Sheam (Acanthopagrus latus) Fish

In this experiment as illustrated in FIG. 4, the fish weight was 399±80.73 g, the water temperature was 25.5° C. and the dissolved oxygen (DO) in the anesthetic and recovery chambers was between 5.5 and 6.32 mg/l. There was no mortality observed in the concentrations between 20 to 60 ppm even after 96-h post recovery. At 80 ppm, there was 50% mortality in the tested population.

### Experiment (4)

### Anesthetic Effect of Thyme, Cinnamon and Oregano Oils Tested on Fingerlings of Sobaity (Sparidentex hasta) Fish at 23° C.

Twenty fish, with body weight range of 285-308 g, were used for each concentration of the four treatments. The data in FIG. 4 shows that all the used concentrations were effective and there was a correlation between concentration and time. The dissolved oxygen (DO) in the anesthetic and recovery chambers was between 5.5 and 6.32 mg/l. No mortality was observed.

### Experiment (5)

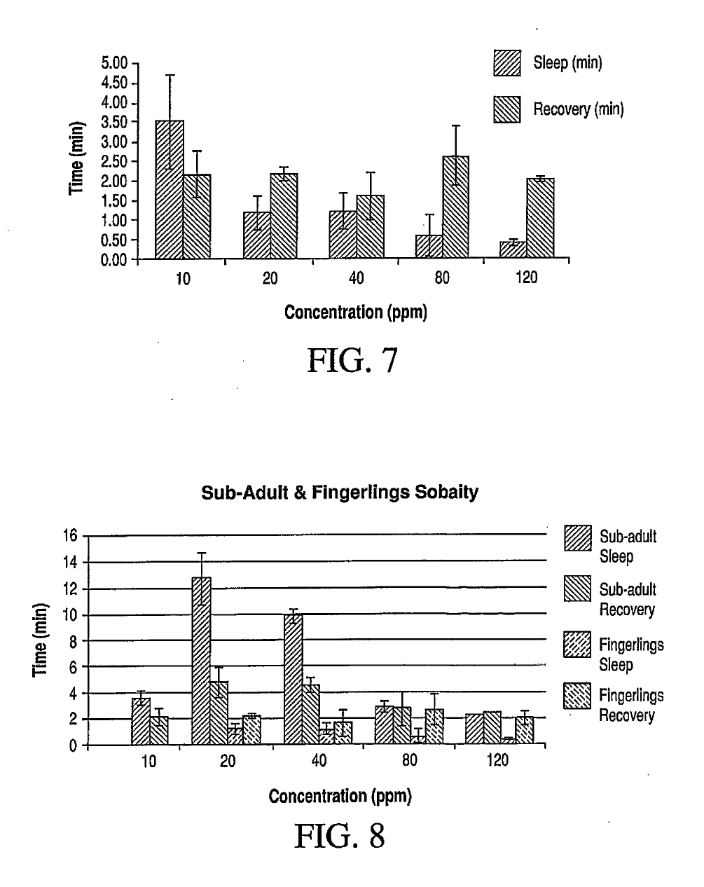

### Anesthetic Effect of Thyme Oil Tested on Fingerlings and Sub-Adult Sobaity (Sparidentex hasta) 23° C. NOTE: FIGS. 5-8

Sub-adult sobaity fish, 255.6±23.54 g, were exposed to concentrations of thyme oil 10, 20, 40, 80 and 120 ppm. The effects of different concentrations of thyme oil on fish were monitored until 10 min and the behavioral changes in the fish were noted. The water temperature was 27° C. and the dissolved oxygen (DO) in the anesthetic and recovery chambers was between 5.5 and 6.32 mg/l. There was no mortality observed in the concentrations between 40 to 120 ppm even after 96-hours post recovery.

### Experiment (6)

### Lethal Dose for 10 min.

Four concentrations of thyme oil (40, 80, 120 and 160 ppm) were tested for ten mm on sobaity with body weight of 141.88±77.45 g and sheam fish with body weight of 40.05±10.31 g.

TABLE 2
Effects of Different Concentrations of Thyme Oil as Anesthetic for sobaity
(Sparidentex hasta) fingerlings at 23.5° C.

| Concentration (ppm) | Lethal Time (min) | % of Dead Fish |
|---|---|---|
| 40 | 10 | 60 |
| 80 | 10 | 100 |
| 120 | 10 | 100 |
| 160 | 10 | 100 |

TABLE 3
Effects of Different Concentrations of Thyme Oil as Anesthetic for Sheam
(Acanthopagrus lotus) fingerlings at 22.7° C.

| Concentration (ppm) | Lethal Time (min) | % of Dead Fish |
|---|---|---|
| 40 | 10 | 10 |
| 80 | 10 | 50 |
| 120 | 10 | 60 |
| 160 | 10 | 90 |

### Experiment (7)

### Lethal Time of Thyme Oil

Sobaity fish (141.88±77.45 g) were kept in the optimum chosen dose, 10 ppm, of thyme oil. Similarly, sheam fish (40.05±10.31 g) were tested at 20 ppm. All fish were kept in the anesthetic for 10 min and no mortality has been reported in both cases.

A concentration 40 ppm of thyme oil has been tested on both species for the lethal time. Sobaity with body weight of 141.88±77.45 g and sheam with body weight of 40.05±10.31 g. At 0.25 min 50% of sobaity fish were dead, while 60% of sheam were dead after 0.26 min.

### Experiment (8)

### Oils Residues in Fish Tissue

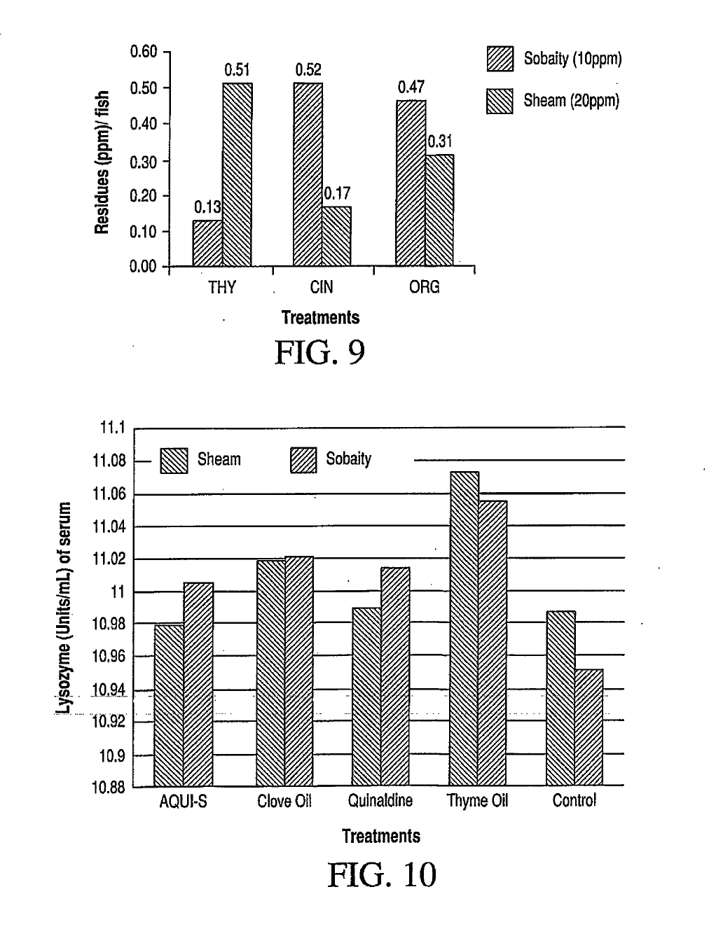

As illustrated in FIG. 9, the primary chemical residue in fillet tissue from all exposed fish was thyme oil, Cinnamon (Cinnamomum zeylanicum) and oregano oil (Origanum vulgare) concentrations in the 0-h sample groups were 0.13, 0.52, 0.47 ppm/fish (129.08±1.38 g) respectively in sobaity and 0.51, 0.17 and 0.31 ppm/fish (41.21±8.92 g) respectively in sheam. Thyme oil consists mainly of thymol. On the other hand, oregano oil consists mainly of carvacrol (40-70%) and thymol, while cinnamon oil consists mainly of (65 to 75% of the oil) is cinnamaldehyde.

### Experiment (9)

### In-Vitro Tests of the Stress Effect of Usine Different Treatments on Fish

Serum lysozyme differed significantly with the EN applied and with the species of fish used. Thyme produced the highest lysozyme activity compared to the others. The ANOVA and the pair-wise comparisons of the treatment means indicated that the thyme oil produced the best effects in reducing the bacterial load after treatment.

### A. Lysozyme Assay

Please refer to FIG. 10 wherein serum lysozyme levels (units/nil) in treated fish against different anesthetics are illustrated.

B. Serum Protein Assay (BCA method)

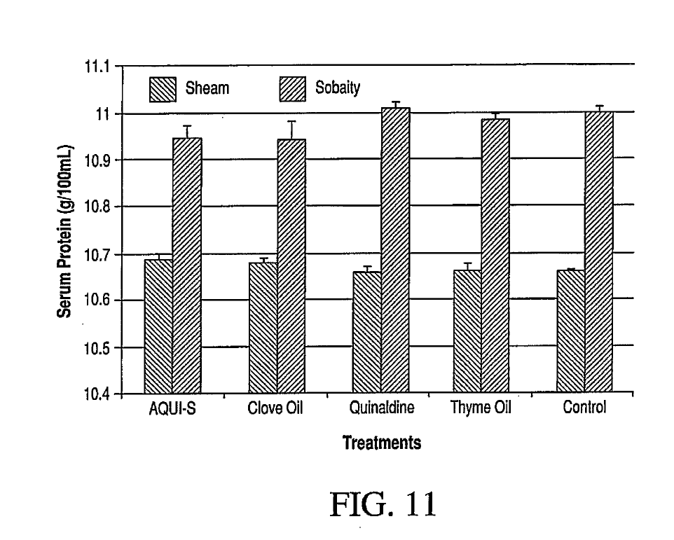

FIG. 11 illustrates a pair-wise comparison of serum protein levels (g/100 ml) in treated fish against different anesthetics.

### C. Thiobarbituric Acid Reactive Substances (TRARS) Assay.

TABLE 4
Serum TBARS Assay in Different Treatments and their Relative
Activity (RA) in Comparison to Control Serum.

| | Sheam | Sobaity |
|---|---|---|
| Treatment | MDA nmol/mL (RA) | MDA nmol/mL (RA) |
| Aqui-S | 0.0262 ± 0.001 (56.95) | 0.0209 ± 0.002 (45.38) |
| Clove Oil | 0.0242 ± 0.001 (52.61) | 0.0187 ± 0.003 (40.59) |
| Quinaldene | 0.0410 ± 0.002 (89.13) | 0.0365 ± 0.003 (79.27) |
| Thyme Oil | 0.0120 ± 0.002 (26.09) | 0.0141 ± 0.003 (30.63) |
| Control | 0.0460 ± 0.001 | 0.0460 ± 0.004 |

RA = 1-(Treatment MDA-Control MDA/Control MDA) X 100

### Experiment (10)

### Evaluating the Effect of the Optimum Anesthetic Doses of E0s and Chemicals on the Fish Blood Parameters

Immediately post exposure to 40 ppm of Eos and chemicals, blood samples were collected from 10 fish, sheam and sobaity, for each concentration. Blood samples were analyzed at a private medical laboratory. Changes in the haematology (HGB, HCT, NEUT, LYMPH, MONO, EO, Cortisol and Glucose) of the tested fish were recorded.

TABLE 5
Effects of 20 ppm of Thyme, Cinnamon and Oregano Oils
on Haematology of Sheam (Acanthopagrus lotus).

| Sheam | Control | Thyme | Cinnamon | Oregano |
|---|---|---|---|---|
| WBCs | 207.40 ± 10.00 | 200.20 ± 11.20 | 180.10 ± 3.50 | 181.13 ± 9.28 |
| RBCS | 3.30 ± 0.30 | 3.40 ± 0.20 | 2.60 ± 0.10 | 2.94 ± 0.18 |
| HGB | 8.40 ± 0.90 | 9.10 ± 0.50 | 6.60 ± 0.00 | 7.50 ± 0.75 |
| HCT | 44.40 ± 5.20 | 46.50 ± 6.70 | 36.50 ± 0.30 | 42.17 ± 3.07 |
| NEUT | 12.00 ± 2.40 | 12.30 ± 1.00 | 14.00 ± 1.00 | 10.00 ± 3.00 |
| (%) | | | | |
| LY- | 80.20 ± 2.70 | 80.80 ± 1.00 | 79.00 ± 1.00 | 83.67 ± 3.51 |
| MPH | | | | |
| (%) | | | | |
| MONO | 3.80 ± 0.80 | 4.00 ± 0.80 | 4.00 ± 0.00 | 3.33 ± 0.58 |
| (%) | | | | |
| EO (%) | 4.00 ± 1.00 | 3.80 ± 1.00 | 3.00 ± 0.00 | 3.00 ± 0.00 |
| Cortisol | 12.50 ± 16.70 | 1.90 ± 1.00 | 14.80 ± 4.30 | 7.14 ± 10.80 |
| (nmol/ | | | | |
| L) | | | | |
| Potas- | 2.20 ± 0.20 | 2.10 ± 0.20 | 1.90 ± 0.00 | 1.80 ± 0.1 |
| sium | | | | |
| (nmol/ | | | | |
| L) | | | | |
| Choles- | 6.60 ± 0.70 | 6.70 ± 0.30 | 5.50 ± 0.00 | 4.91 ± 0.07 |
| terol | | | | |
| (nmol/ | | | | |
| mL) | | | | |
| Cal- | 3.90 ± 0.40 | 3.90 ± 0.10 | 3.70 ± 0.00 | 3.81 ± 0.14 |
| cium | | | | |
| (nmol/ | | | | |
| L) | | | | |
| Glucose | 1.00 ± 0.60 | 1.80 ± 0.87 | 2.10 ± 0.50 | 5.84 ± 0.39 |
| (nmol/ | | | | |
| L) | | | | |

TABLE 6
Effects of 20 ppm of Thyme, Cinnamon and Oregano Oils on
Haematology of Sobaity (Sparidentex hasta).

| Sobaity | Control | Thyme | Cinnamon | Oregano |
|---|---|---|---|---|
| WBCs | 207.30 ± 12.50 | 211.00 ± 9.50 | 194.50 ± 11.70 | 204.80 ± 9.50 |
| RRCs | 3.60 ± 0.20 | 3.70 ± 0.30 | 3.40 ± 0.20 | 3.70 ± 0.30 |
| HGB | 9.70 ± 0.40 | 9.80 ± 0.30 | 8.90 ± 0.50 | 9.60 ± 0.20 |
| HCT | 51.80 ± 3.60 | 44.60 ± 5.30 | 48.40 ± 5.20 | 45.40 ± 5.00 |
| NEUT | 14.40 ± 2.30 | 11.40 ± 3.80 | 14.00 ± 4.00 | 12.70 ± 1.50 |
| (%) | | | | |
| LY- | 78.20 ± 2.90 | 80.00 ± 4.40 | 79.00 ± 3.60 | 81.30 ± 2.10 |
| MPH | | | | |
| (%) | | | | |
| MONO | 3.60 ± 0.90 | 4.40 ± 0.50 | 3.30 ± 0.60 | 3.00 ± 0.00 |
| (%) | | | | |
| E0 (%) | 3.80 ± 0.80 | 4.20 ± 0.80 | 3.70 ± 0.60 | 3.70 ± 0.60 |
| Cortisol | 53.60 ± 18.10 | 15.90 ± 11.40 | 335.20 ± 141.70 | 37.70 ± 8.30 |
| (nmol/ | | | | |
| L) | | | | |
| Potas- | 4.30 ± 0.70 | 2.90 ± 0.10 | 3.00 ± 0.30 | 3.40 ± 0.10 |
| sium | | | | |
| (nmol/ | | | | |
| L) | | | | |
| Choles - | 7.10 ± 0.30 | 6.00 ± 0.20 | 5.50 ± 0.90 | 7.90 ± 0.40 |
| terol | | | | |
| (nmol/ | | | | |
| L) | | | | |
| Calcium | 3.90 ± 0.70 | 4.20 ± 0.50 | 3.50 ± 0.10 | 4.20 ± 0.10 |
| (nmol/ | | | | |
| L) | | | | |
| Glucose | 4.90 ± 0.40 | 8.60 ± 0.10 | 10.10 ± 1.30 | 8.60 ± 0.50 |
| (nmol/ | | | | |
| L) | | | | |

### Experiment (11)

### Anesthetic Effect of Thyme on Shrimp (Penaeus semisulcatus)

Twenty shrimp with body weight of 10.71+3.09 g were tested under 100 ppm of thyme oil till 10 min. Some shrimp lost their balance and regained it back Some were on the side with collapsed front legs. The back legs never stopped moving (which is a sign for total sleep). The shrimp were easy to catch and to handle. A concentration of 200 ppm of thyme has been tested and all tested, shrimp (10.71+3.09 g) went on their sides (sign of anesthesia induction) with no balance at 7.25-8.15 min. The back legs never stopped moving.

### II. Antibacterial Effect of the Essential Oils

### Experiment (12)

### Bactericidal Effect Immediately after Complete Sleep

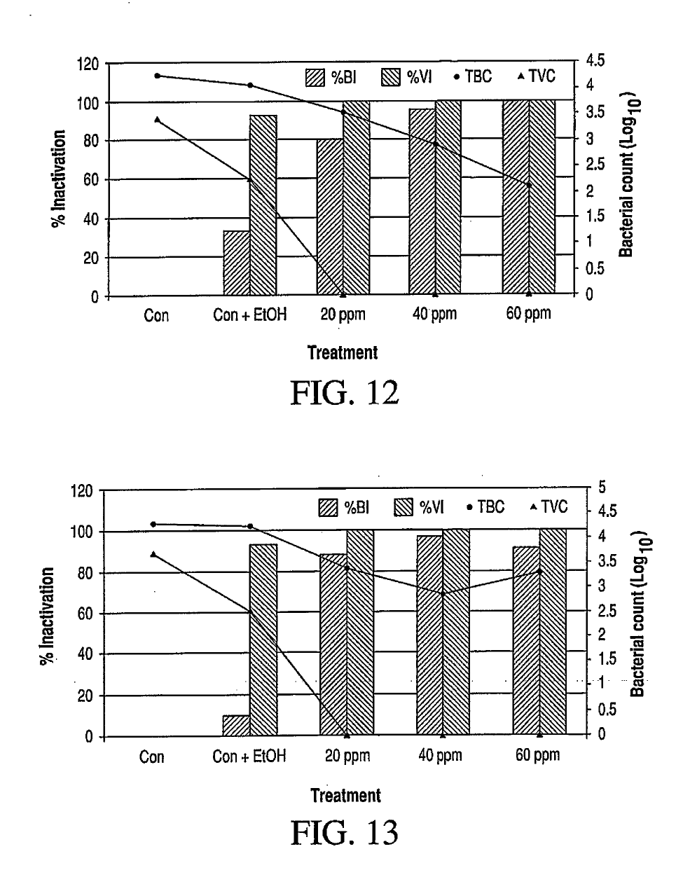

With reference to FIG. 12, the total bacterial in vibrio (log₁₀) counts (TBC and TVC) and their percent inactivation after treatment with thyme oil are illustrated in FIGS. 12 and 13. Swabs from the treated fish were collected directly after anesthesia and cultured for bacterial count to investigate the antibacterial effect of each treatment just after total anesthesia. The FIGS. 12 and 13 show the antibacterial effects of thyme oil, clove oil, quinaldine and AQUI-S, after treating sobaity fish.

### Experiment (13)

### Bactericidal Assay of Different E0s Against Important Aquatic Pathogenic Bacteria

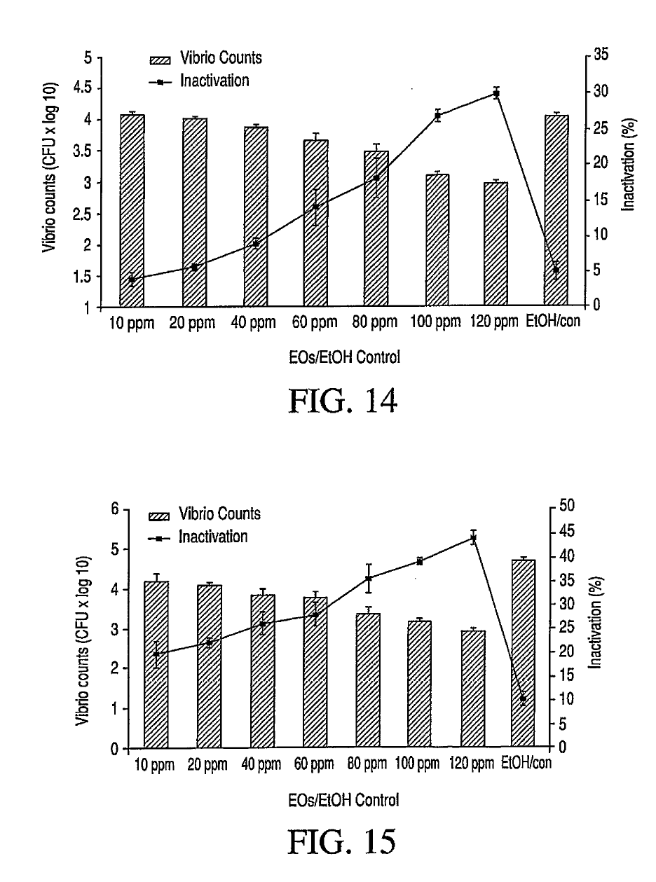

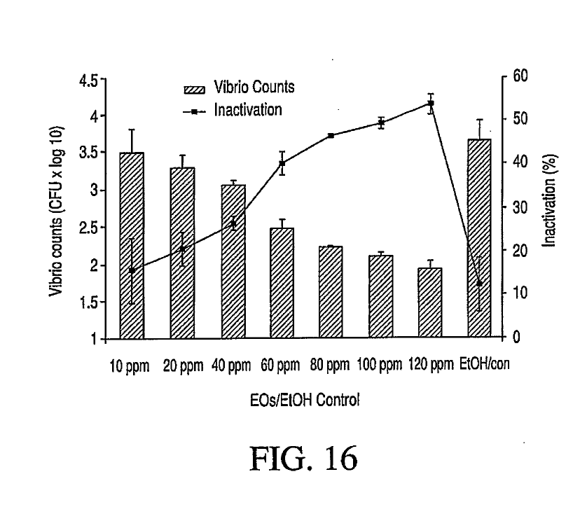

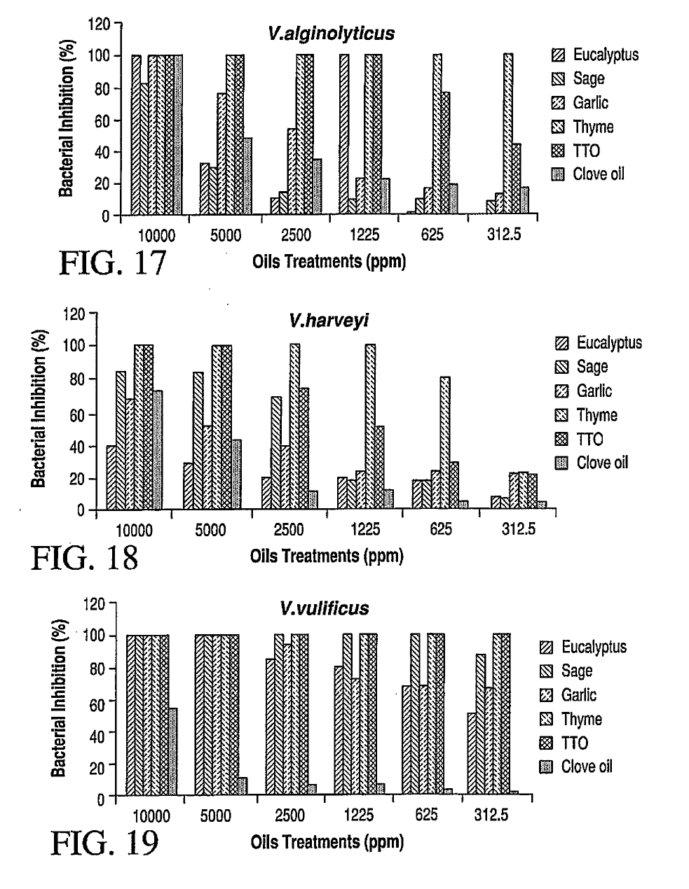

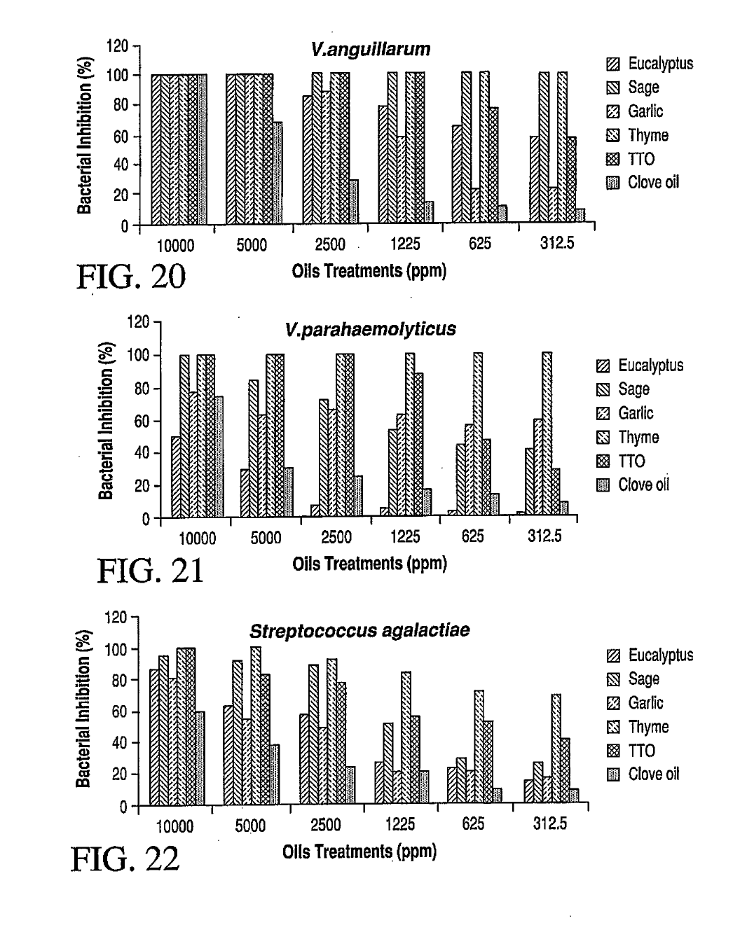

Different concentrations (10000, 5000, 2500, 1225, 625, 312.5 ppm) of the selected E0s (sage, garlic, thyme, TTO, eucalyptus, cinnamon and clove) were tested against the selected bacteria. Bactericidal assay was carried out using the micro drop plating method employing doubling dilutions of various E0s. Complete bactericidal activity was achieved with thyme in a majority of bacterial species tested and with all concentrations of the E0s. V. alginolyticus, V. anguillarum, V parahaemolyticus and V. vulnificus were completely killed at all levels tested with thyme oil. Refer to FIGS. 14, 15, 16, 17-22, and 28.

TABLE 8
Overall Bactericidal Activity of Different Concentrations of Each
EO Tested against Important Aquatic Pathogenic Bacteria.

| Concentration | Bactericidal Activity (%) | | | | | |
|---|---|---|---|---|---|---|
| (ppm) | EO | SO | GO | TO | TTO | CO |
| 10000 | 79.44 | 93.64 | 87.47 | 100.00 | 100.00 | 76.78 |
| 5000 | 58.89 | 81.59 | 72.47 | 100.00 | 97.13 | 40.24 |
| 2500 | 44.39 | 73.79 | 65.62 | 98.57 | 91.68 | 22.09 |
| 1250 | 35.71 | 55.70 | 43.37 | 97.14 | 82.34 | 16.00 |
| 625 | 29.84 | 50.41 | 35.27 | 91.97 | 63.63 | 11.00 |
| 313 | 22.41 | 45.61 | 34.00 | 82.02 | 49.22 | 08.71 |

EO = Eucalyptus
SO = Sage
GO = -Garlic
TO = Thyme
TTO = Tea tree
CO = Clove

### Experiment (14)

### Inhibitory Activity of E0s Tested at Different Concentrations Against Important Aquatic Pathogenic Bacterial Species

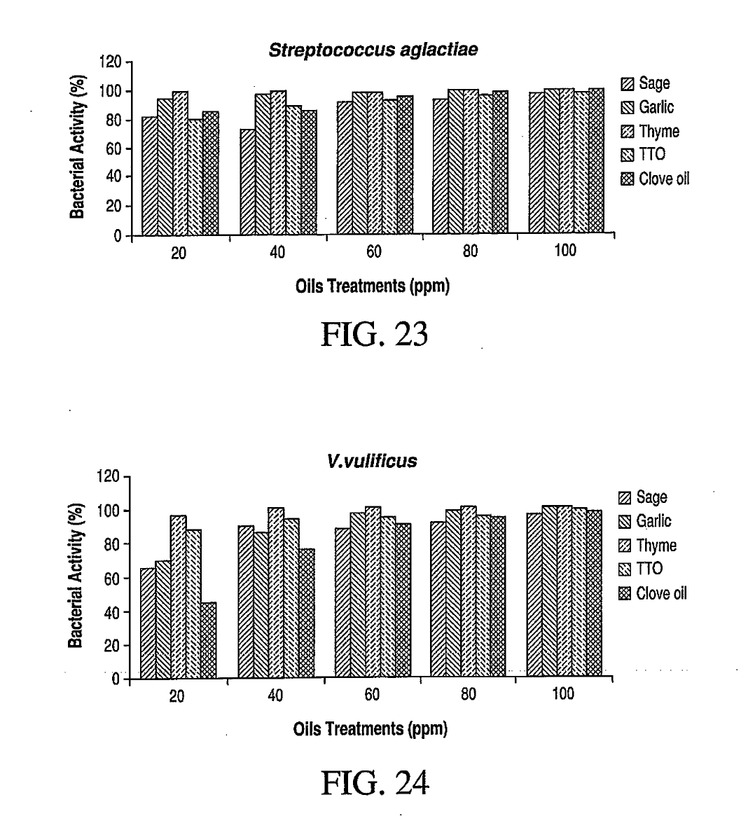

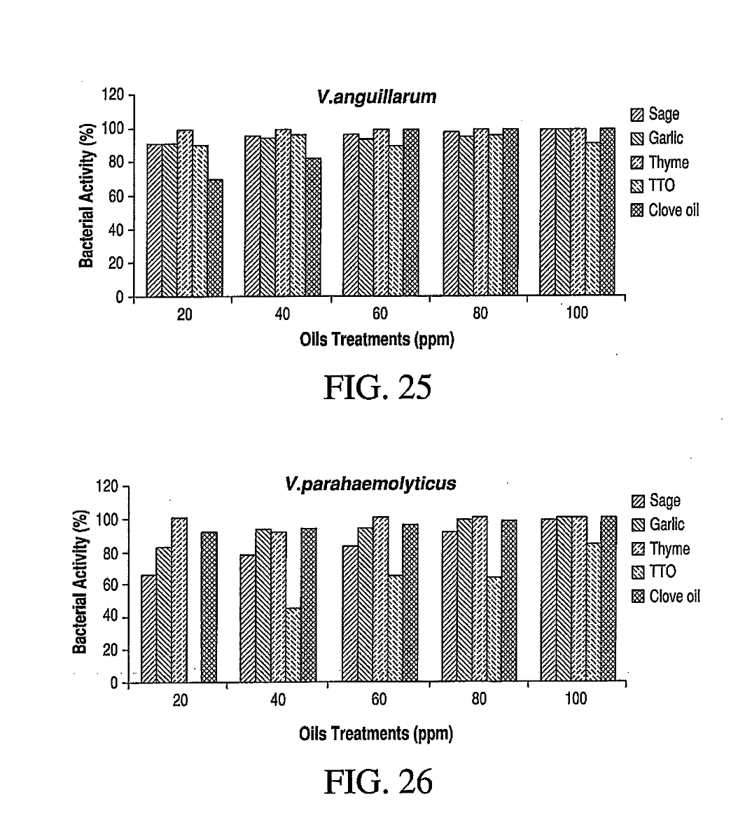

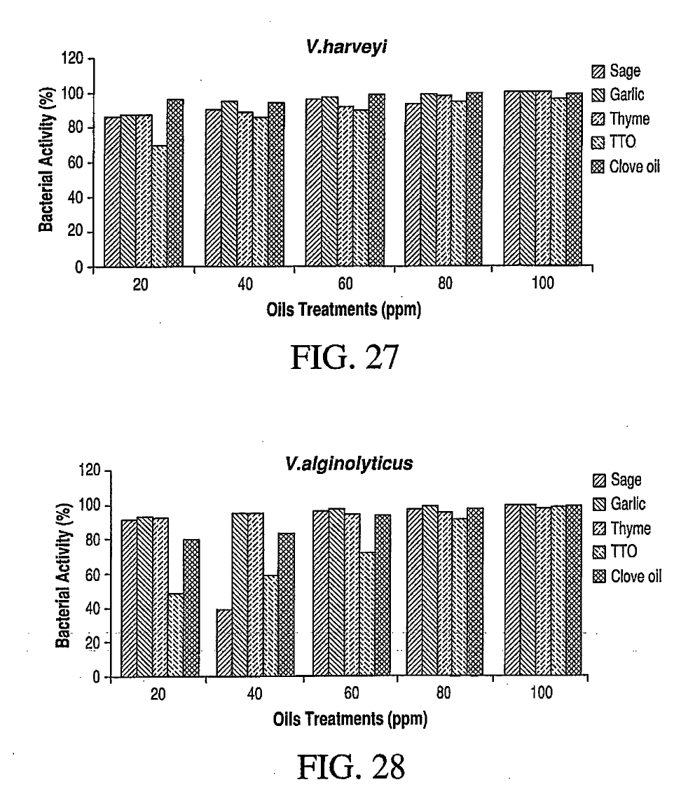

Different concentrations (20, 40, 60, 80 and 100 ppm) of the selected E0s (sage, garlic, thyme, TTO and clove) were tested against the selected bacteria, V. vulnificus, V anguillarurn, V. parahaemolyticus, V. vulificus, V. harveyi, V. alginolyticus, and S. aglactiae. Of all the EOs, thyme oil produced the best bacterial inhibition with 97.3% growth inhibition among all the bacterial species tested followed by garlic (95.02%). Refer now to FIGS. 23-28.

TABLE 7
Quantitative Inhibitory Activity of Thyme Oil (TO) Tested at Different
Concentrations against Important Aquatic Pathogenic Bacterial Species.

| | V. alginoorticus | | V. anguillarum | | V. harveyi | | V. parahaemolyticus | | V. vulnificus | | Streptococcus aglactiae | |
|---|---|---|---|---|---|---|---|---|---|---|---|---|
| (ppm) | % | CFU | % | CFU | % | CFU | % | CFU | % | CFU | % | CFU |
| 20 | 93.0 | 9.1 × 10⁶ | 98.5 | 3.0 × 10⁷ | 86.6 | 1.8 × 10⁷ | 99.5 | 1.7 × 10⁷ | 96.9 | 2.2 × 10⁶ | 99.0 | 2.1 × 10⁵ |
| 40 | 95.6 | 5.7 × 10⁶ | 99.0 | 2.0 × 10⁷ | 88.1 | 1.6 × 10⁷ | 92.1 | 1.9 × 10⁷ | 99.1 | 6.5 × 10⁵ | 99.0 | 2.3 × 10⁵ |
| 60 | 94.8 | 6.8 × 10⁶ | 99.6 | 8.1 × 10⁵ | 91.5 | 1.2 × 10⁷ | 99.3 | 1.7 × 10⁶ | 99.7 | 2.0 × 10⁵ | 99.5 | 1.1 × 10⁵ |
| 80 | 95.6 | 5.7 × 10⁶ | 99.9 | 2.1 × 10⁶ | 98.0 | 2.8 × 10⁶ | 99.4 | 1.3 × 10⁶ | 99.9 | 1.1 × 10⁵ | 99.8 | 4.5 × 10⁴ |
| 100 | 97.2 | 3.6 × 10⁵ | 100.0 | 7.5 × 10⁵ | 99.0 | 1.4 × 10⁶ | 100.0 | 1.8 × 10⁵ | 100.0 | 1.0 × 10⁴ | 100.0 | 3.6 × 10³ |

HSD (Concentrations) = 19.05 (at P = A.05)
HSD (Bacteria) = 21.95 (at P = 0.05)
Difference between the treatment means for concentrations (Con) and for bacterial species (Bact) exceeding the HSD ‘q’ are significantly different from one another at = 0.05

### Experiment (15)

### Treatment of Natural Infection Using E0s

The sheam fish were infected naturally with bacteria causing the swelling and clouding of the eye, possibly due to a sudden change in water quality supporting the bacterial infection. Treatment was given to control this infection due to mixed etiology. The fish were treated for 5 d with ppm of Eos (TTO, thyme, garlic, sage, clove and eucalyptus). The positive result was the

| | | HGB | FICT | | LYMPH | MONO | EO | Colstrrol | Glocose |
|---|---|---|---|---|---|---|---|---|---|
| EC's | N | (g/d1) | CYO | NEUT | (A) | (%) | (%) | (mmol/1) | (mmol/1) |
| Control | 10 | 10.40 ± 0.98 | 44.56 ± 6.80 | 9.00 ± 1.00 | 79.40 ± 2.41 | 4.80 ± 0.84 | 4.60 ± 0.55 | 6.12 ± 1.32 | 1.63 ± 0.86 |
| Thyme | 10 | 9.40 ± 0.85 | 43.35 ± 8.37 | 15.40 ± 1.14 | 75.40 ± 2A1 | 4.40 ± 0.55 | 3.80 ± 0.45 | 6.06 ± 0.79 | 0.40 ± 0.46 |

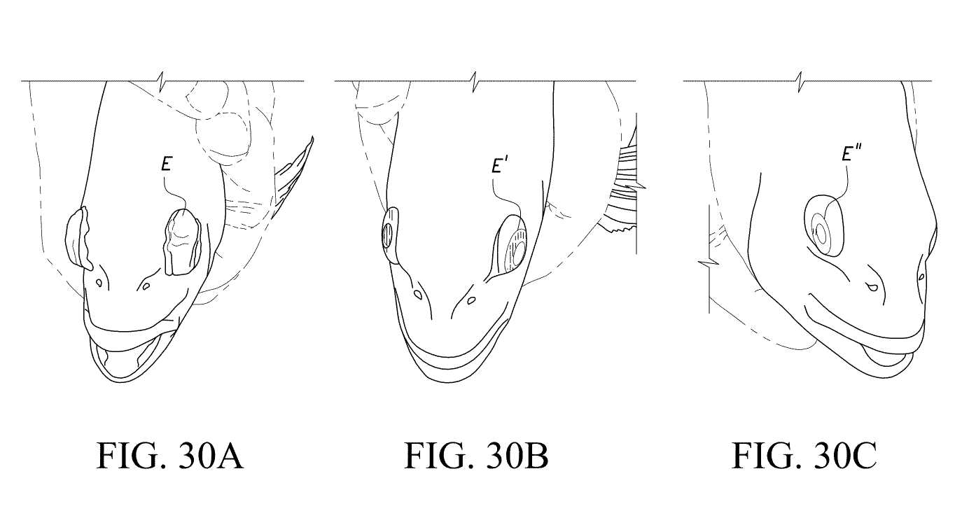

nearly the same on all the treatments. FIGS. 30A-30C illustrate bacterial eye infection in Shaeim (Acanthopagrus latus) showing healing effects of natural oils before treatment (E), during treatment (E′), and after treatment, (E″), respectively.

The shrimp suffered from another natural infection (browning of the gills) caused by bacteria, and was treated for 5-d with 20 ppm of 6 EOs (TTO, sage, garlic, thyme, clove and eucalyptus) for 30 min After the 5-days treatment, improvement showed reducing the browning in the gills.

TABLE 8
The Effect of Five Days of Treatment with 20 ppm of EOs on the Blood
Parameters of Shaeim (Acanthopagrus latus).
HCT = Hematocrit HGB = Hemoglobin LYMPH = Lymphocyte MONO = Monocyte EO = Eosinophil NEW = Neutrophil

| | | | | | | | | | |
|---|---|---|---|---|---|---|---|---|---|
| TTO | 10 | 9.95 ± 0.97 | 48.63 ± 1.72 | 15.75 ± 0.96 | 75.00 ± 0.82 | 5.00 ± 0.00 | 4.20 ± 0.84 | 6.36 ± 1.17 | 0.48 ± 0.27 |
| Garlic | 10 | 9.17 ± 0.81 | 45.47 ± 1.32 | 15.60 ± 3.78 | 74.80 ± 4.15 | 4.60 ± 0.55 | 4.60 ± 0.55 | 5.59 ± 0.33 | 0.24 ± 0.18 |
| Sage | 10 | 9.75 ± 0.22 | 47.95 ± 1.00 | 18.67 ± 0.88 | 77.80 ± 2.85 | 4.40 ± 0.24 | 4.40 ± 0.24 | 5.62 ± 0.14 | 0.73 ± 0.24 |
| Clove | 10 | 8.92 ± 0.95 | 46.65 ± 2.86 | 16.20 ± 2.39 | 75.80 ± 249 | 4.20 ± 0.84 | 3.80 ± 1.10 | 5.58 ± 0.51 | 1.07 ± 0.76 |

### Phenol Oxidase and Bacterial Killing Ability of Haemolymph.

All the treated shrimps, irrespective of the type of E0, produced elevated phenol oxidase (PO) levels and enhanced microbicidal activity.

TABLE 9
Hemolymph of P. semisulcatus Exposed to Different E0s (20 ppm)
against V. alginolyticus.

| E0s | CFU | Bactericidal (%) | Mean APO | Mean RPO |
|---|---|---|---|---|
| Control | 1.6 × 10⁸ | 0.00 | 24.00 ± 9.50 | 0 |
| Thyme | 3.4 × 10⁷ | 78.75 | 84.60 ± 2.20 | 60.60 ± 9.50 |
| Clove | 3.6 × 10⁷ | 77.5 | 71.60 ± 3.70 | 47.60 ± 9.60 |
| TTO | 3.0 × 10⁷ | 81.25 | 86.60 ± 9.10 | 62.60 ± 17.10 |
| Garlic | 4.5 × 10⁷ | 71.87 | 77.80 ± 5.10 | 53.80 ± 9.20 |
| Eucalyptus | 7.2 × 10⁷ | 55.00 | 106.20 ± 16.80 | 82.20 ± 25.80 |
| Sage | 3.8 × 10⁷ | 76.25 | 62.00 ± 3.50 | 38.00 ± 12.90 |

HSD (APO) = 29.27 (at P = 0.05);
HSD (RPO) = 16.37 (at P = 0.05)
APO = Absolute phenol oxidase;
RPO = Relative phenol oxidase

### Experiment (16)

### Effect of Different EOs on Uronetna sp.

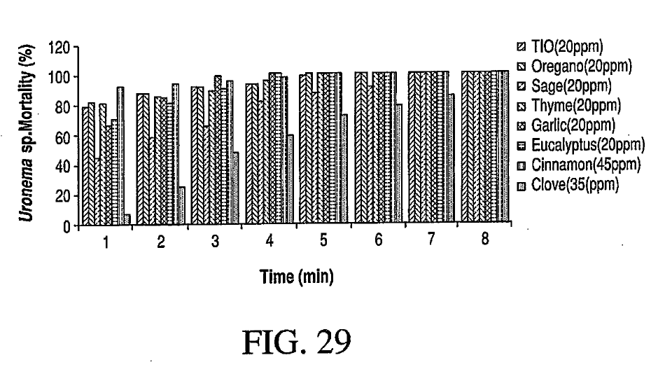

Referring now to FIG. 29, the effects of the concentrations (5, 10, 15, 20, 25 ppm) of E0s were tested on Uronema cells. All the oils showed a positive effect to kill the parasite plate (2). Known volumes of Uronema culture suspension with a fixed parasite cell concentration was subjected to the varying concentrations of each of the EOs, and the response of the live parasite cells to the EOs were recorded on a time scale. Changes in the cell morphology and activity were recorded until lysis or death of cells. Cessation of ciliary motility was considered as cell death. All concentrations had five replicates laid out on a 24-well tissue culture polystyrene plate having 500 pi well capacity. A minimum of 100 cells of Uronema was counted in each well for calculating lethal concentrations. All EOs showed effective anti-parasitic effect.

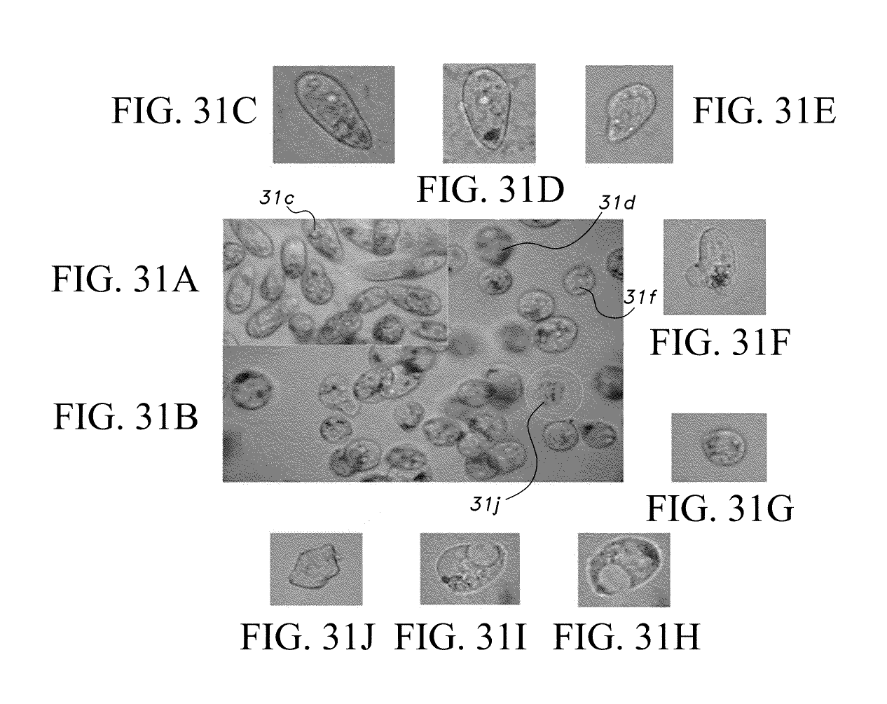

The stages of Uronerna sp. cell depredation following exposure to most of the E0s were as follows: FIG. 31A illustrates normal shaped cells; and the other stages of cell depredation are shown in area FIG. 31B.
Normal shape cells were irritated in their straightforward movement, then they slowed down, followed by upside down movement forward being static. FIGS. 31A, 31C and 31c.
Blebbing of the cell with bubble-like protrusions. FIG. 31 D, 31d,
Blubbing or bulging of the cell. FIG. 31E.
Raindrop shape cell formation. FIG. 31F, 31f.
   Finally, rupturing of cell membrane FIGS. 31G-I, followed by the cell lysis FIG. 31 J, 31j,

While the invention has been disclosed in connection with its preferred embodiments it should be recognized that changes and modifications may be made therein without departing from the scope of the claims.

1-7. (canceled)

8. An anesthetic and therapeutic composition for use in aquaculture consisting of:
an essential oil selected from the group consisting of thyme oil, cinnamon oil, oregano oil and mixtures thereof;
ethanol and sea water;
wherein said essential oil and ethanol are provided in the ratio of one part essential oil to five parts ethanol and wherein the amount of sea water provides a composition having between 10 ppm to 60 ppm essential oil plus a dissolved oxygen content between 5.00 and 6.5 mg/l.

9-10. (canceled)

11. A method for treating fish with an anesthetic and therapeutic composition for reducing stress during routine procedures and as a therapeutic for bacterial and parasitic infections, said method comprising the steps of:
providing a first tank and a mass of water disposed in said tank and maintaining said water in said first tank at a temperature of about 20-27° C. and providing a quantity of an anesthetic and therapeutic composition for reducing stress during routine procedures and as a therapeutic for bacterial and parasitic infections, wherein the composition comprises:
a) an essential oil selected from the group consisting of thyme oil, cinnamon oil, oregano oil and mixtures thereof;
b) ethanol and water;
wherein said essential oil and ethanol are provided in the ratio of one part essential oil to five parts ethanol and wherein the amount of water provides a composition having between 10 ppm to 60 ppm essential oil plus a dissolved oxygen content between 5.00 and 6.5 mg/l;
providing a second tank of water at a temperature of about 20-27° C.;
introducing a number of fish into said first tank and maintaining said fish in said first tank for a period of ten minutes until each of said fish shows signs of sleep induced by said anesthetic; and
transferring said fish after ten minutes exposure to said second tank for a period of 96 hours.

12. A method for treating fish with an anesthetic and therapeutic composition according to claim 11 wherein said contents of said first and second tanks are each maintained with a dissolved oxygen content of between about 5.0 to about 6.5 mg/l.

13. (canceled)

14. A method for treating sobaity (Sparidentex pasta) fish, sheam (Acanthopalatus) fish and shrimp with an anesthetic and therapeutic composition for reducing stress during routine procedures and as a therapeutic for bacterial and parasitic infections, said method consisting of:
providing a first tank and a mass of sea water disposed in such first tank and maintaining sea water in said first tank at a temperature of between about 20° C. and 27° C. and providing a mass of essential oils selected from the group consisting of thyme oil, cinnamon oil, oregano oil and mixtures thereof; and ethanol and wherein the mixture provides a ratio of one part essential oil to five parts ethanol and wherein the mass of sea water contains between about 10 ppm to 60 ppm of said essential oil;
providing a second tank and a mass of sea water disposed in said second tank and maintained at a temperature between about 20° C. and 27° C.;
exposing said fish to the essential oil in said first tank for a period of about 10 minutes;
removing said fish from said first tank and placing said fish in said second tank and allowing said fish to remain therein for 96 hours of observation and detection of any mortality; and
testing said fish for changes in hematology (HGB, HCT, NEUT, LYMPH, MONO, EO, Cortisol and Glucose).

15. A method for treating sobaity (Sparidentex hasta) fish, sheam (Acanthopalatus) fish and shrimp with an anesthetic and therapeutic composition for reducing stress during routine procedures and as a therapeutic for bacterial and parasitic infections according to claim 14 in which said mass of essential oil is thyme oil.
